(19) United States
(12) Patent Application Publication
(10) Pub. No.: US 2005/0049495 A1
(43) Pub. Date: Mar. 3, 2005

(54) Remote assistance for medical diagnostic ultrasound

(75) Inventors: Thilaka S. Sumanaweera, Los Altos, CA (US); Stefan Josef Anton Schmitz, Milpitas, CA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/654,509

(22) Filed: Sep. 3, 2003

(51) Int. Cl. A61B 8/00

(52) U.S. Cl. 600/437

(57) ABSTRACT

Methods and systems for assistance in medical diagnostic ultrasound imaging are provided. Users are provided access to a vast knowledge base of ultrasound information, such as the knowledge base acquired by ultrasound imaging system manufacturers. Manufacturers may have more up-to-date ultrasound technology, processing and information. Users from various locations are provided access to this knowledge at a central location. For example, factory certified expert processing of ultrasonic data is provided via the Internet. Image processing, analysis and interpretation are provided by a computer or by a human at the central location. The expertise represented by the computer or human processing is then provided back to the various locations. Ultrasound imaging system users distributed throughout different facilities benefit from the processing, analysis and interpretation of ultrasound data provided by the central location.

### BACKGROUND

The present invention relates to medical diagnostic ultrasound imaging. In particular, assistance in medical diagnostic processing is provided.

Medical diagnostic ultrasound imaging systems are used locally. The patient is placed adjacent to the imaging system. A sonographer or medical professional scans the patient. The data acquired by scanning the patient is then processed and displayed locally, such as at the imaging system or an associated workstation. The processes used to generate the image, quantify aspects of the image or identify features of the image are performed by the imaging system processors. Any diagnosis is typically performed by the sonographer or medical professional in viewing the image. However, the sonographer or medical professional may fail to understand nuances of the processes used to generate the image or have out-of-date software, resulting in less ideal imaging.

Diagnostic assistance may be provided by an additional sonographer or medical professional viewing the imaging. The image is stored at the local ultrasound imaging system or stored in a network within a facility for later display at the imaging system or a work station within the facility. U.S. Pat. No. 6,322,505 teaches transmission of image information to a remote location for viewing at the remote location. In addition to diagnostic assistance through a second opinion or viewing of the image, additional processing is performed at the remote location.

A national digital mammography archive has been developed, at least in part, by a plurality of university related hospitals for storing X-ray or MRI-based mammograms. A central memory or depository stores mammograms from the various institutions. Images, reports and other information related to each individual patient are accessible at any of various network locations. Computer aided diagnosis, such as provided by using various filtering and neural network algorithms, may assist in identifying possible microcalcifications or lesions for further study. The computer aided diagnosis is provided at the repository for access from the various facilities.

### BRIEF SUMMARY

The present invention is defined by the following claims, and nothing in this section should be taken as a limitation on those claims. By way of introduction, the preferred embodiments described below include methods and systems for assistance in medical diagnostic ultrasound imaging. Users are provided access to a vast knowledge base of ultrasound information, such as the knowledge base acquired by ultrasound imaging system manufacturers. Manufacturers may have more up-to-date ultrasound technology, processing and information. Users from various locations are provided access to this knowledge at a central location. For example, factory certified expert processing of ultrasonic data is provided via the Internet. Image processing, analysis and interpretation are provided by a computer or by a human at the central location. The expertise represented by the computer or human processing is then provided back to the various locations. Ultrasound imaging system users distributed throughout different facilities benefit from the processing, analysis and interpretation of ultrasound data provided by the central location.

In a first aspect, a method for remote assistance in local medical diagnostic ultrasound imaging is provided. Ultrasound image data is acquired at a local location. The ultrasound image data is transmitted from the local location to a remote location. The remote location is in a different facility than the local location. Processed data responsive to the ultrasound image data is then received at the local location from the remote location.

In a second aspect, a method for assistance in medical diagnostic ultrasound imaging is provided. Ultrasound image data is received from two different remote locations. The two different locations are at different facilities. The received ultrasound image data is processed at a third location different than the first and second remote locations. The processed data is then transmitted to the remote location from which the original ultrasound image data was received. The processed data may also or alternatively be transmitted to a fourth remote location different from the first, second and third locations.

In a third aspect, a system for assistance in medical diagnostic ultrasound imaging is provided. A first medical diagnostic ultrasound imaging system is at a first location. A second medical diagnostic ultrasound imaging system is at a second location at a different facility than the first location. A server is operable to receive ultrasound imaging information from the two different imaging systems. The server is operable to process the ultrasound imaging information and transmit the processed result to the respective imaging systems.

Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for assistance in medical diagnostic ultrasound imaging; and

FIG. 2 is a flow chart diagram of one embodiment of a method for assistance in medical diagnostic ultrasound imaging.

### DETAILED DESCRIPTION OF THE DRAWINGS AND PRESENTLY PREFERRED EMBODIMENTS

Ultrasonic data is acquired at each of a plurality of facilities at the same or different times. Using a medical diagnostic ultrasound imaging system, an associated workstation, and local network or other processor, the acquired ultrasound data is submitted to a remote server through a network, such as the Internet. The remote server receives ultrasound imaging information from the different facilities. The remote server is operated by an ultrasonic equipment manufacturer or other entity having an ultrasound knowledge base. Any of various processes are requested either automatically or through user input. In response to either a direct message, such as an inserted request code, an implicit message based on the type of ultrasound imaging data transmitted to the server or a predefined agreement between the local facility and the server, processing by the server is requested. The server processes the ultrasound data, such as automatically processing the ultrasound data or processing the ultrasound data in conjunction with sonographer or medical professional input. The results of the processing are then transmitted back to the appropriate local facilities, such as transmission over the Internet. The results may then be used at each of the various local facilities as part of the diagnosis using ultrasound images. The results may also be sent to a facility other than the one from which the data were originally received. The communications and processes happen in real-time during an imaging session or at different times for later diagnosis assistance. All or part of the communication can be encrypted to ensure privacy.

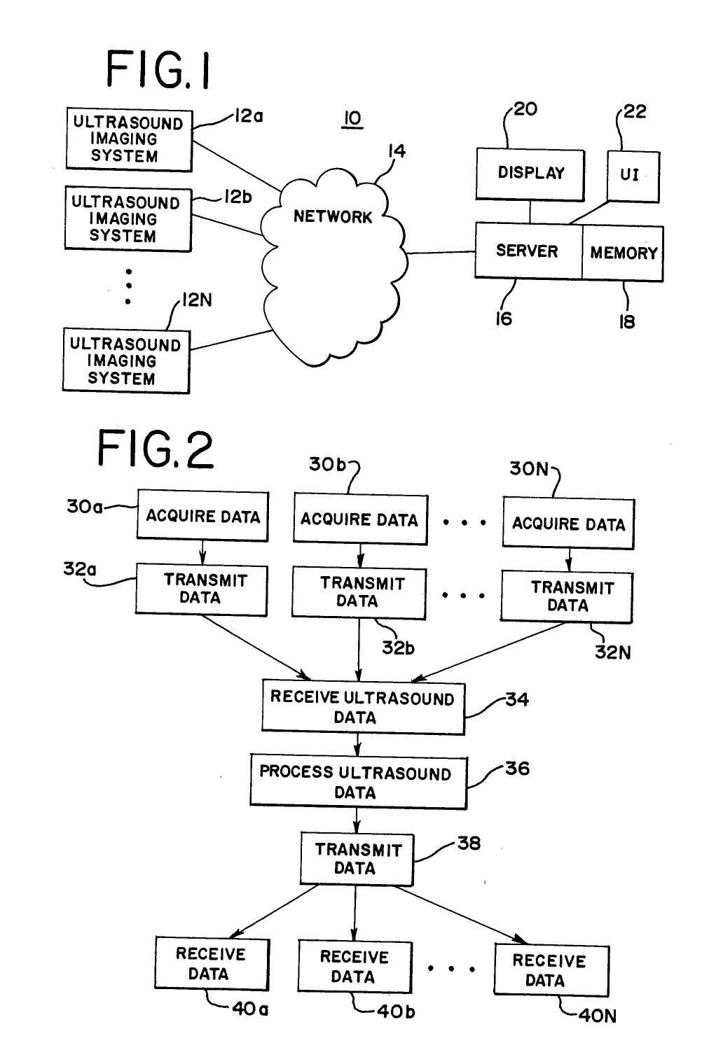

FIG. 1 shows one embodiment of a system 10 for assistance in medical diagnostic ultrasound imaging. Pluralities of medical diagnostic ultrasound imaging systems 12a through 12N connect through a network 14 to a server 16. The server 16 has an associated memory 18, display 20 and user interface 22. Additional, different or fewer components may be provided for the system 10, such as no display 20 or user interface 22.

The medical diagnostic ultrasound imaging systems 12 include a transmit beamformer, a transducer, a receive beamformer, a detector, a scan converter and a display. For example, an Antares or Sequoia system manufactured by Siemens Medical Solutions, USA, Inc. is provided. Medical diagnostic imaging systems manufactured by other manufacturers may be used. In alternative embodiments, one or more of the ultrasound imaging systems 12 are workstations for further processing or display. In yet other alternative embodiments, one or more of the ultrasound imaging systems 12 includes a local area network, such as a network at a hospital, and an associated server and memory for accessing ultrasound image data. In yet other alternative embodiments, one or more of the ultrasound imaging systems 12 are laptop computers, personal digital assistants (PDA) or cellular phones. Different imaging systems may be used for different ones of the systems 12a through 12N.

Two or more ultrasound imaging systems 12 are provided, such as fifty or more. Each ultrasound imaging system is associated with a different location or facility. For example, each imaging system 12 is associated with a clinic or hospital in a different building, in a different city and/or different state. In one embodiment, the system 10 is adapted to work world wide so that any of the imaging systems 12 are of various locations and facilities throughout the world. Country or region specific implementations may also be provided. In one embodiment, one or more of the ultrasound imaging systems 12 include a plurality of imaging systems at a single facility with or without associated work stations and local area networking. Others of the ultrasound imaging systems 12 represent an individual medical diagnostic ultrasound imaging system, such as a free-standing cart system at a clinic. Yet others of the ultrasound imaging systems 12 represent portable, such as hand-held or movable, ultrasound systems, laptop computers, personal data assistants (PDAs) and cellular phones that are connectable with the network 14. The ultrasound imaging systems 12 could be located in mobile vehicles, such as emergency response vehicles, airborne crafts, seaborne crafts and submersible crafts. Any combination of all of the same types, different types, same manufacturer, different manufacturer or other characteristics are provided for the ultrasound imaging systems 12 for different locations and associated different facilities.

The network 14 is a computer network, such as a wide area network, the Internet, the telephone, wired, land line, satellite, television, videophone, courier, wireless, combinations thereof or other now known or latter developed communications network. In one embodiment, the network 14 includes a plurality of networks connected together through one or more communications devices. In other embodiments, the network 14 comprises the telephone system or other direct link between each ultrasound imaging system 12 and the server 16. The network 14 may include routers, switches, or other devices for transmitting information from one or more of the ultrasound imaging systems to the server 16 and from the server 16 to one or more of the ultrasound imaging systems 12. In one embodiment, the network 14 is a high speed network with sufficient bandwidth for transmitting high resolution ultrasound imaging in a manner of seconds. The communications network 14 electrically connects the ultrasound imaging systems 12a through 12N to the server 16. For example modem, wireless, Ethernet or other electrical connections between each of the imaging systems 12 and the network 14 are provided. The server 16 connects to the network 14 using any of the above described electrical connections. All or parts of the transmissions may be encrypted to ensure privacy.

The ultrasound image information received at the server 16 or processed by the server 16 is stored in the memory 18. The memory 18 is a local memory for the server 16, a bank of RAM memory, a removable media (e.g., tapes, optical storage, reel or other now known or latter developed devices), combinations thereof or other now known or latter developed memory devices. The memory 18 acts as a repository or archive for ultrasound image information received at different times from different ultrasound imaging systems 12. The memory 18 is controlled by the server 16 for storing information prior to processing or storing processed results. In one embodiment, the memory 18 is scalable so that as additional ultrasound images are saved, more information may be further accepted. Alternatively, the memory 18 stores information for a limited period.

The server 16 is a processor, group of processors, application specific integrated circuit, computer, workstation, bank of servers, bank of processors, local area network with distributed processing, mainframe computer, or other know known or later developed devices for processing data and communicating over the network 14. The server 16 is operable to receive ultrasound imaging information from different locations. The server 16 is further operable to process the ultrasound imaging information. The same or different processors are used for receiving and processing the information.

In one embodiment, the server 16 automatically processes the ultrasound imaging information without user input at the server. For example, the server 16 receives ultrasound image information from a plurality of ultrasound imaging systems 12. The same or different processes are performed by the server 16 on the received ultrasound imaging information without human intervention. As another example, one or more processors of the server are operable to process ultrasound imaging information based on a characteristic of other ultrasound images. Received ultrasound imaging information is compared with one or more other ultrasound images associated with a particular diagnosis or feature. The server 16 identifies a similarity or dissimilarity from expected images.

The server 16 is further operable to transmit the process results back to the ultrasound imaging system 12 from which the original ultrasound imaging information was received. The processed results may be transmitted to other ultrasound imaging systems 12 as well or alternatively. Processed information is transmitted in a same or different format with or without the originally received ultrasound information. Since the server 16 transmits the processed results through the network 14 to the one or more ultrasound imaging systems 12, an interactive process is provided. All or parts of the transmission may be encrypted to ensure privacy. Processing is requested by the ultrasound imaging system 12, and processed results are provided back by the server 16.

As an alternative or in addition to automated processing by the server 16, the server 16 includes the display 20 and the user interface 22 for processing at least in part provided in response to user input. The display 20 is a CRT, flat panel, LCD, plasma, projection or other now known or latter developed display device. The display 20 connects with the server 16 through a remote connection or in a location local to the server 16. The display 20 is operable to show images responsive to the ultrasound imaging information received from one or more of the ultrasound imaging systems 12. Alternatively, the server has the capability to print the received ultrasonic data on film, photographic paper or other types of paper.

The user input 22 is a keyboard, mouse, trackball, touchpad, ultrasound imaging system controls or other now known or latter developed user input devices. The user input 22 is operable to receive diagnosis information from a user and provide the diagnosis information to the server 16. For example, one or more images are displayed on the display 20. A sonographer, cardiologist, radiologist or other trained personnel view the image and suggest a diagnosis, an area for further study, a surgical procedure, processing to be performed by the server 16, processing to be performed by one of the ultrasound imaging systems 12, identify borders, identify defects in scanning, or other information. The server may assist the trained personnel by processing the data and suggesting options. The information is provided to the server 16 for either further processing or routing as processed results to the appropriate ultrasound imaging system 12. In one embodiment, a user controls a process implemented by the server 16 or by a different processor associated with the display 20 and a user interface 22. The processed information is then provided to or by the server 16 for transmission to the appropriate ultrasound imaging systems 12.

FIG. 2 shows one embodiment on a method for remote assistance in medical diagnostic ultrasound imaging. The method uses the system 10 of FIG. 1 or a different system. Additional, different or fewer acts than shown in FIG. 2 may be used.

In acts 30a through 30N, ultrasound image data is acquired at various local locations. The ultrasound image data is acquired at a same or different time at each of the locations. The locations are associated with different facilities, such as being remote from each other. “N” represents any integer number, indicating a plurality of individual remote locations for acquiring ultrasound image data.

The ultrasound image data includes an image in any of various formats. For example, a linear, sector, or Vector® or other now known or latter developed image is acquired. The transducers used can be 1D, 1.25D, 1.5D, 1.75D, 2D or 3D. The ultrasonic images acquired can be 1D, 2D, 3D or 4D. The image is acquired using any now known or latter developed detection techniques, such as modes for detecting a second harmonic ultrasound image, a contrast agent image, a continuous wave image, a pulse wave image, a Doppler image, a Doppler and B-mode combination image, a B-mode image, an M-mode image or combinations thereof. The ultrasound image data includes information for a single image or for a sequence of images. For example, a sequence of Doppler ultrasound images is acquired to show change in flow over time. The sequence of images represents a heart cycle or other time period, such as acquiring 30 or more images over a one second time period. The ultrasound image information is acquired as video information from a scan converted output of an ultrasound imaging system in one embodiment, but detected information in a polar coordinate format, predetected information, received beamformed information or other ultrasound imaging data may be acquired for transmission.

The ultrasound image data is acquired during a discrete imaging session. A discrete imaging session corresponds to an examination of a patient using one or multiple modes of ultrasound imaging. A typical imaging session lasts from 10 minutes to an hour, but may be longer or shorter. The patient may not be required to stay still during this period of time, unlike CT, MRI or mammography. The patient is radiated with sound pulses, a non-ionizing radiation in contrast to mammograms. An imaging session corresponds with a patient examination at a given time or appointment. In alternative embodiments, the ultrasound image data represents information acquired at multiple different imaging sessions for the same or different patients.

In acts 32a through 32N, the ultrasound image data is transmitted from the various local locations to a remote, central location. The central location is in a different facility than all or a subset of the various local locations. The ultrasound image information is transmitted electronically, such as over a computer network or via video-transmission. All or parts of the transmission can be encrypted to ensure privacy. Alternatively, the information is transmitted on a movable storage device, such a compact disk, memory diskette, tape or other now known or latter developed portable storage device. The stored data can also be encrypted. For electronic transmission, the data is transmitted in one of any now known or latter developed formats, such as an HTML, JPEG, MPEG, DICOM, XML, or other now known or latter developed computer network communications format for video or image data. For example, the ultrasound imaging information is transmitted as a plurality of packets of data in a TCP/IP format.

In one embodiment, the ultrasound image information includes labeling information, such as a designator of the location or system used to acquire the data, patient information, diagnostic history, sonographer, medical personnel, and/or other information. In one embodiment, parameters identifying various processing that has occurred in the acquisition of the data are transmitted with the data, such as disclosed in U.S. Pat. No. 6,322,505, the disclosure of which is incorporated herein by reference. Additional information transmitted with the ultrasound image information may be a requested process. For example, a known coding system is used for requesting a specific process or processes to be performed remotely on the ultrasound image information. Any specific or general codes associated with processing or common questions for diagnosis are included. In alternative embodiments, additional request or other information is not provided, and the remote location determines processes to be performed through contractual agreements, the type of image information received, or a request communicated through other channels.

The transmitted ultrasound image information includes any of various types of images, such as Doppler ultrasound images. One or more images may be contained in a given transmission, such as transmitting a sequence of images. Alternatively, a sequence of images or a single image is transmitted through multiple discrete transmissions. The transmissions are performed as the image information is acquired, such as configuring prior to acquisition an ultrasound imaging system to generate images locally as well as transmit information to the remote server. Alternatively, images are labeled for transmission as they are acquired or soon after acquisition. In yet other embodiments, the ultrasound image information is transmitted after an imaging session. By transmitting the ultrasound image information to the server or a remote location, the knowledge base associated with the remote location (e.g. an ultrasonic equipment manufacturer's knowledge base and processes) is provided for processing the ultrasound image information in addition to diagnosis provided at the location where the information is acquired.

The knowledge base is provided as a free service for purchasers of ultrasound medical diagnostic equipment in one embodiment. In other embodiments, a contracted price based on time, number of images or other objective criteria is charged to the facility or entity associated with the acquisition of the ultrasound data. Alternatively, insurance companies or other entities are billed.

In act 34, the transmitted ultrasound image data is received at the remote or central location. The data is received from each of a plurality of different other locations or facilities. The ultrasound image data is in a same or different format as was transmitted. If the data has been encrypted, it is decrypted for processing. The received ultrasound image data is queued for processing in the order in which the data is received. In alternative embodiments, ultrasound image data from one or more of the plurality of facilities is provided with a priority. The received ultrasound image data from a plurality of remote locations is queued for subsequent sequential or parallel processing. For example, the server 16 receives a plurality of ultrasound image information data sets over a computer network over a short or long time period. Once a complete data set is received, the data set is labeled with an identifier for a position in a queue for further processing.

In act 36, the ultrasound image data received from a plurality of different locations or facilities is processed. Any of various processing may be provided, such as processing to identify tumors, detect borders, classify tissues, perform ejection fraction measurements or other cardiac quantifications, filter the image information for enhancing one or more qualities, providing a diagnosis, providing a second opinion, providing surgical planning based on the image, providing suggestions for further imaging to be performed, or other processes.

In one embodiment, the received ultrasound image data is processed automatically with a server or other processor. The automatic processing occurs without user input between the reception of the ultrasound information and the transmission of the processed result. For example, the processor extracts the desired process to be performed from the received ultrasound data as a function of a coded header or a type of ultrasound information received. The process is then performed with a processor, such as providing a particular type of filtering, quantification, or other processor implemented algorithm. For example, a more up-to-date, efficient, complex, thorough or otherwise different process is performed by the central location than is available at the locations where the ultrasound data is acquired. New technologies for processing and the state of the art performance of the processing may be more likely available to the central location than to individual remote locations. Financially and/or intellectually disadvantaged ultrasonic scanning centers or other facilities capable of acquiring ultrasound data may take advantage of more advanced processes performed at the centralized location.

In an alternative embodiment, some user input is provided at the central location for performing the processing. For example, a user views the acquired ultrasound image data or an image resulting from the receive ultrasound image data and indicates a type of processing to be performed by a processor. As another example, the user indicates that a requested process should be performed. As yet another example, user performs some or all of the processing by entering diagnostic information. Ultrasound images responsive to the received ultrasound image data are displayed. The user at the central location enters diagnostic information with a user input. For example, a diagnostic conclusion is entered. As another example, highlighting for a particular location of the image with or without further annotation is entered. As yet another example, a mere acknowledgment of, agreement with or disagreement with another opinion is entered. As another example, surgical planning information, such as the type of equipment, how to use the equipment, when to use the equipment and where to use the equipment during surgery is entered. The entered information is processed data provided in response to the received ultrasound image information. Sonographers or medical professionals are able to assist medical professionals at other locations. Alternatively, engineers or others familiar with ultrasound processes are able to distinguish process artifacts from areas of medical concern.

In one embodiment using automated or human controlled processes, an index of similarity or relational information is provided. For example, an archive of a plurality of ultrasound images is stored in the memory 18. In another embodiment, data derived from the ultrasound images is stored in the memory 18. The similarity between a received ultrasound image or data derived from the received ultrasound image and the stored image information or data is calculated or otherwise indicated. The similarity index or indices may indicate a similarity with a healthy tissue or a similarity with unhealthy tissue. For example, the knowledge of a plurality of doctors, sonographers or others is replicated in a relational database. The similarity index is provided by processing the image information to determine its relationship to the knowledge base. The index of similarity may include an assignment of probability. For example, a tissue of interest is identified as more or less probable to be a tumor. Since the central location has access to information from the other facilities or from a knowledge base of experts, such as from a manufacturer, a relational database may be used to provide similarity information not otherwise available to an individual remote location. The index of similarity is based on correlation, neural network, filters, or other now known or latter developed algorithms for identifying a relationship of one set of information with other sets of information.

In act 38, the processed data is transmitted from the central location to the requesting facility or the facility associated with the acquisition of the data. The transmission may be encrypted, in whole or in part, to ensure privacy. In other embodiments, the processed data is transmitted to a different facility or to a plurality of facilities. In one embodiment, the processed data is provided to the system used to acquire the ultrasound image data. The processed data is then displayed by the system for diagnosis. For example, the response to the request at a local facility is provided during the same imaging session as when the data was acquired. Further scanning for the completion of diagnosis is then performed during the imaging session based in part on the reprocessed data provided from the central location. Diagnosis information, such as for a second opinion or other human input information, is provided as the processed data and is transmitted for further use. In alternative embodiments, the processed data is printed on printable media and/or converted into voice and played back to the receiver.

In acts 40a through 40N, the processed data responsive to the originally acquired and transmitted ultrasound image data is received at one or more of the local locations from the central or remote location. The receivers can be ultrasound systems, viewing workstations, laptop computers, PDAs, telephones or cellular phones or other devices discussed above for the systems 12. The processed data is received in a same format or a different format than as the same ultrasound image information transmitted in acts 32. In one embodiment, the originally acquired and transmitted ultrasound image data is provided with the processed data. Alternatively, the processed data is provided without the originally transmitted ultrasound image data. The processed data is provided over a same communications network or through a different communications route. All or parts of the communication may be encrypted to ensure privacy. Processed data received is image data, diagnostic data, quantities or other processed information. For example, the processed data includes diagnosis recommendations, such as an initial diagnosis by a processor or person at the central location, a second opinion or surgical planning information. As another example, a filtered version of the ultrasound image data is provided as the processed data. As yet another example, a quantification representing a measurement from the ultrasound image data is provided. As another example, a region of interest, tissue of interest, time within a sequence of interest or combinations thereof is provided as the processed data. In yet another embodiment, the processed data may be derived from Doppler data. In one embodiment, the processed data is received during the same imaging session in which the original ultrasound image information was acquired in acts 30. In alternative embodiments, the processed data is received after the imaging session.

Using the method of FIG. 2 or the system of FIG. 1, ultrasonic data is acquired by a user. The ultrasonic data is submitted to a remote server or central location. Processed results are obtained from the remote server or central location. The user then uses the processed results for further medical diagnosis or other treatment of a patient. The individual user or facility has the advantage of input from the central or remote location with a larger or different knowledge base. Proprietary or public information about the way ultrasonic data is acquired and processed may be used to provide additional information not otherwise available to the user. Knowledge base acquired due to a manufacturer's or other centralized location's global presence or experience may provide more efficient, more ideal or different processing of the data than would be performed by the user or the user's local system. New technologies for processing and diagnosis may be available to the central location not otherwise available to individual users or facilities. Facilities not able to purchase state of the art processing software and systems or with reduced opportunity for diagnostic assistance may take advantage of the centralized location processing for further diagnosis and processing not otherwise available. Where the central location is provided by a specific manufacturer of ultrasound imaging systems, the manufacturer may become known not just for systems for acquiring data, but also for processing data in an advantageous way. The manufacturer provides an overall solution for medical diagnostic ultrasound imaging.

In one embodiment, the server 16 sends back imaging parameters and/or pre-sets to the local imaging system or other originator of the data. For example, software operable to program the imaging system 12 with a set of parameters not currently available is provided to the local imaging system. As another example, suggested settings to be programmed, adjusted or set by the user of the local system are provided. The local user scans the patient, sends the resulting image data to the server 16. The server 16 sends back processed results to implement a rescan by changing the imaging parameters. Since ultrasound imaging is real-time compared to mammograms, this process can be performed during an imaging session.

While the invention has been described above by reference to various embodiments, it should be understood that many advantages and modifications can be made without departing from the scope of the invention. For example, different ultrasound image processes may be performed at the local, remote or central facilities. As another example, different facilities may communicate over different types of networks to the same central location or server.

It is therefore intended that the foregoing detailed description be regarded as illustrative rather than limiting, and that it be understood that it is the following claims, including all equivalents, that are intended to define the spirit and the scope of this invention.

1. A method for remote assistance in local medical diagnostic ultrasound imaging, the method comprising:
(a) acquiring ultrasound image data at a first local location;
(b) transmitting the ultrasound image data from the first local location to a remote location, the remote location in a different facility than the first local location; and
(c) receiving from the remote location processed data responsive to the ultrasound image data at one of: the first local location and a second local location different than the first local location and the remote location.

2. The method of claim 1 wherein (c) comprises receiving diagnosis recommendations as the processed data.

3. The method of claim 1 wherein (c) comprises receiving a filtered version of the ultrasound image data as the processed data.

4. The method of claim 1 wherein (c) comprises receiving quantification data as the processed data.

5. The method of claim 1 further comprising:
(d) automatically processing the ultrasound image data with a processor at the remote location, the result of (d) being the processed data.

6. The method of claim 1 further comprising:
(d) entering data from a user input at the remote location, the data being the processed data.

7. The method of claim 1 wherein (a) comprises acquiring a sequence of Doppler ultrasound images and wherein (b) comprises transmitting the sequence of Doppler ultrasound images.

8. The method of claim 1 wherein (a) comprises acquiring an image selected from the group of: a second harmonic ultrasound image, a continuous wave image, a pulsed wave image, a Doppler image, a Doppler and B-mode image, a B-mode image, and an M-mode image, wherein (b) comprises transmitting the image.

9. The method of claim 1 wherein (a) comprises acquiring the ultrasound image data during an imaging session, wherein (b) comprises transmitting the ultrasound image data as (a) is performed, and wherein (c) comprises receiving the processed data during the imaging session.

10. The method of claim 1 wherein (c) comprises receiving from the remote location the processed data responsive to the ultrasound image data at the first local location.

11. The method of claim 1 wherein (a) comprises acquiring ultrasound image data representing a three-dimensional volume.

12. The method of claim 1 further comprising:
(d) encrypting one of: the acquired ultrasound data prior to (b) and the processed data prior to (c).

13. A method for assistance in medical diagnostic ultrasound imaging, the method comprising:
(a) receiving first ultrasound image data from a first remote location;
(b) receiving second ultrasound image data from a second remote location, the first remote location at a different facility than the first remote location;
(c) processing the first and second ultrasound image data at a third location different than the first and second remote locations; and
(d) transmitting first and second processed data responsive to (c) from the third location.

14. The method of claim 13 further comprising:
(e) receiving other ultrasound image data from a plurality of other remote locations;
wherein (c) comprises processing the first, second and other ultrasound image data.

15. The method of claim 13 wherein (a) and (b) comprise receiving at a server over a network and wherein (c) comprises automatically processing the first and second ultrasound image data with the server.

16. The method of claim 15 wherein (c) comprises automatically processing without user input after (a) and (b) and before (d).

17. The method of claim 13 wherein (c) comprises transmitting the first and second processed data to the first and second remote locations, respectively.

18. The method of claim 13 wherein (c) comprises processing data representing a three-dimensional volume.

19. The method of claim 13 further comprising:
(e) encrypting the processed data prior to (d).

20. The method of claim 13 wherein (a) and (b) comprise receiving at a server over a network and wherein (c) comprises:
(c1) displaying first and second images responsive to the first and second ultrasound data, respectively; and
(c2) entering first and second diagnosis information in response to (c1) with a user input;
wherein (d) comprises transmitting the first and second diagnosis information.

21. The method of claim 13 wherein (c) comprises generating an index of similarity.

22. A system for assistance in medical diagnostic ultrasound imaging, the system comprising:
a first medical diagnostic ultrasound imaging system at a first location;
a second medical diagnostic ultrasound imaging system as a second location, the second location at a different facility than the first location;
a server operable to receive first and second ultrasound imaging information from the first and second medical diagnostic ultrasound imaging systems, respectively, the server operable to process the first and second ultrasound imaging information and operable to transmit processed results to one of: the first medical diagnostic ultrasound imaging system, the second medical diagnostic ultrasound imaging system, a third medical diagnostic ultrasound imaging system different than the first and second medical diagnostic ultrasound imaging systems, and combinations thereof.

23. The system of claim 22 further comprising:
a display connected with the server, the display operable to show first and second images responsive to the first and second ultrasound imaging information, respectively; and
a user input operable to provide diagnosis information to the server as the processed results.

24. The system of claim 22 wherein the server is operable to automatically process the first and second ultrasound imaging information without user input at the server.

25. The system of claim 22 further comprising:
a memory connected with the server, the memory operable to store a plurality of ultrasound images;
wherein the processor is operable to process the first ultrasound imaging information based on a characteristic of the plurality of ultrasound images.

26. The system of claim 22 further comprising:
at least fifty additional medical diagnostic ultrasound imaging systems at individually remote locations; and
a communications network electrically connecting the additional medical diagnostic ultrasound imaging systems to the server.

27. The system of claim 22 wherein the processor is operable to encrypt the processed results.
